(19) United States
(12) Patent Application Publication
(10) Pub. No.: US 2010/0143461 A1
(43) Pub. Date: Jun. 10, 2010

(54) PALONOSETRON FORMULATION

(76) Inventors: Ben-Zion Solomon, Petach Tikva (IL); Zvika Doani, Tel-Mond (IL)

Correspondence Address: KENYON & KENYON LLP, ONE BROADWAY, NEW YORK, NY, 10004, US

(21) Appl. No.: 12/633,563

(22) Filed: Dec. 8, 2009

Related U.S. Application Data
(60) Provisional application No. 61/201,262, filed on Dec. 8, 2008.

(51) Int. Cl. A61K 31/44; A61K 9/48; A61K 9/28

(52) U.S. Cl. 424/452; 424/451; 424/456; 424/474; 514/296

(57) ABSTRACT

The present invention provides solid oral formulations of palonosetron or salts thereof.

This application claims the benefit of U.S. Provisional Application No. 61/201,262, filed Dec. 8, 2008.

### FIELD OF THE INVENTION

The invention relates to oral formulations of anti-emetic 5-HT₃ antagonist drugs. In particular, the present invention provides stable solid oral formulations of palonosetron or salts thereof.

### BACKGROUND OF THE INVENTION

Palonosetron [CAS Registry No. 119904-90-4] is a selective serotonin 5HT₃ receptor antagonist. Palonosetron is marketed in the form of its hydrochloride salt [CAS Registry No. 135729-62-3]. The chemical name for this compound is: (3aS)-2-[(3S)-1-azabicyclo[2.2.2]oct-3-yl]-2,3,3a,4,5,6-hexahydro-1-oxo-1H-benz-[de]isoquinoline hydrochloride or 2-(quinuclidin-3(S)-yl)-2,3,3a(S),4,5,6-hexahydro-1H-benz[de] isoquinolin-1-one hydrochloride, and its molecular formula is represented as:

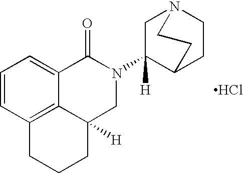

Palonosetron and its synthesis was first disclosed in U.S. Pat. No. 5,202,333. Palonosetron hydrochloride is marketed in the US under the trade name Aloxi® by Helsinn Healthcare. Aloxi® is available as single 0.25 mg or 0.075 mg injections for intravenous use. This formulation is indicated for the prevention of acute and delayed nausea and vomiting associated with initial and repeat courses of moderately emetogenic cancer chemotherapy, and for the prevention of nausea and vomiting associated with initial and repeat courses of highly emetogenic cancer chemotherapy. Aloxi® i.v. is also indicated for the prevention of postoperative nausea and vomiting (PONY) for up to 24 hours following surgery.

WO2004/067005 apparently discloses stable liquid formulations of palonosetron useful for the preparation of injectable and liquid oral medicaments. The authors of WO2004/067005 disclose that intravenous formulations of palonosetron suffer from shelf stability issues. Allegedly, in order to address the stability problems associated with injectable palonosetron formulations, the authors provide solution formulations having a pH range of from about 4.0 to about 6.0, and optionally excipients including mannitol and a chelating agent.

US 2008/0152704 (WO2008/049552) appears to disclose soft gel capsule formulations consist of a soft gelatin-based outer capsule shell having a low oxygen permeability, and a capsule filling which is a continuous lipophilic phase containing palonosetron dissolved in an aqueous component, which is miscibilized or homogenized in the lipophilic phase by a surfactant. The authors of this publication report that the disclosed soft-gel capsules are stable and provide the desired bioavailability upon oral ingestion. According to the authors, the continuous liquid phase provides ease of processing and composition uniformity. Further, the authors disclose that a soft outer shell having a particular oxygen permeability is preferred due to the ability of the shell to hold liquid and to resist oxygen transmission. US 2008/0152704 discloses representative gel-cap formulations that incorporate an antioxidant such as butylated hydroxyanisole, in order to provide the required stability.

Palonosetron is required to be a fast-acting drug and thus must have a high bioavailability. In prior art formulations and publications the high bioavailability, allegedly, has been achieved by providing the drug as a liquid preparation, for example as an intravenous injection or other liquid preparation such as an encapsulated liquid. In view of the known stability problems associated with liquid formulations containing palonosetron and its salts, there is a need to provide stable dosage forms, preferably without the use of one or more excipient or chemical agent to prevent oxygen mediated degradation of palonosetron in the dosage form. Preferably, it would be desirable to provide a dosage form comprising palonosetron or a pharmaceutically acceptable salt thereof in the solid phase.

One of the main obstacles to the production of solid palonosetron compositions is that palonosetron is a low dose drug (0.25 mg 0.5 mg or 0.75 mg per unit dosage form). This presents a problem because the incorporation of such small quantities of drug into a final dosage form can result in poor content uniformity—i.e. the drug is not uniformly distributed in the dosage form. Another challenge is that the active agent needs to be fast acting, and thus must have a good bioavailability profile. In particular the bioavailability profile should be comparable to that of a liquid or gel formulation.

It would therefore be highly desirable to provide a dosage form containing palonosetron or a pharmaceutically acceptable salt thereof wherein the drug is in the solid phase, and in which the bioavailability of the drug is comparable to that of a liquid formulation. It would further be desirable to provide a formulation of palonosetron or a pharmaceutically acceptable salt thereof that is stable and is easy to manufacture. It would further be desirable to provide a solid palonosetron composition having a good dissolution profile and good blend and/or content uniformity.

### SUMMARY OF THE INVENTION

In a first aspect, the present invention provides a dosage form for oral administration comprising a solid admixture of palonosetron or a pharmaceutically acceptable salt thereof and at least one pharmaceutically acceptable excipient. The dosage form can be a solid dosage form. The formulation is preferably in the form of a filled hard gelatin capsule, a powder, granules, a tablet including an orally disintegrating tablet, or effervescent tablet, wherein the tablets may optionally be coated. Preferably, the dosage form according to the present invention does not contain an antioxidant. In a preferred embodiment the dosage form is a hard gelatin capsule containing a solid admixture of palonosetron or pharmaceutically acceptable salt thereof (preferably palonosetron hydrochloride) and at least one pharmaceutically acceptable excipient, wherein the dosage form does not contain an antioxidant.

In one embodiment, the dosage form of the present invention is in the form of a hard gelatin capsule dosage form comprising, preferably consisting of, (with the wt % based on total weight of the capsule filling, and excluding the capsule shell):
palonosetron hydrochloride in an amount of about 0.1 to about 2.0 wt %, preferably about 0.15 to about 0.32 wt %, preferably about 0.15 to about 0.3 wt % or about 0.18 to about 0.30 wt %, preferably about 0.15 to about 0.25 wt %, more preferably about 0.18 to about 0.25 wt %; wherein the palonosetron hydrochloride is more preferably in an amount of about 0.25 to about 0.29 and especially about 0.25 to about 0.28 wt %;
diluent in an amount of about 60 to about 90 wt %, preferably about 60 to about 85 wt %, preferably about 65 to about 85 wt % or about 65 to about 80 wt %, and most preferably about 75 to about 85% or 75 to about 80 wt %; wherein the diluent can be a mixture of lactose and microcrystalline cellulose, or lactose and mannitol but is, preferably lactose;
binder in an amount of about 1 to about 8 wt %, preferably about 2 to about 6 wt %, more preferably about 4-5 wt %, and preferably wherein the binder is povidone (preferably povidone K-30);
disintegrant in an amount of about 10 to about 25 wt %, or about 8 to about 25 wt % preferably about 12 to about 20 wt % or about 8 to about 20 wt %, and more preferably about 15 to about 18, or about 8 to about 12 wt %, particularly about 10% and preferably wherein the disintegrant is crospovidone, croscarmellose sodium, sodium starch glycolate, or mixtures thereof, particularly a mixture of croscarmellose sodium and sodium starch glycolate; and
lubricant in an amount of about 0.5 to about 2 wt %, preferably about 0.5 to about 1.8 wt %, more preferably about 0.5 to about 1.5 wt % or about 1.0 to about 1.2 wt % or about 1 to about 1.5 wt %, and preferably wherein the lubricant is magnesium stearate or sodium stearyl fumarate, with sodium stearyl fumarate being particularly preferred.

Preferably, the dosage form does not contain any antioxidants.

In a further embodiment, the dosage form is a hard gelatin capsule comprising, preferably consisting of, (with the wt % based on total weight of the capsule filling, and excluding the capsule shell):
palonosetron hydrochloride in an amount of about 0.2 to about 0.35 wt %, preferably about 0.25 to about 0.3 wt % and more preferably about 0.28 wt %;
diluent in an amount of about 75 to about 85 wt %, preferably about 80 to about 85 wt %, and more preferably about 83 wt %;
binder in an amount of about 3 to about 6 wt %, preferably about 4 to about 6 wt %, and more preferably about 5 wt %;
disintegrant in an amount of about 5 to about 15 wt %, preferably about 8 to about 12 wt %, and more preferably about 10 wt %; and
lubricant in an amount of about 1 to about 2.5 wt %, preferably about 1.1 to about 2.4 wt %, and more preferably about 1.5 wt %.

In another embodiment, the dosage form of the present invention may be in the form of an orally disintegrating tablet or effervescent dosage form comprising, preferably consisting of, (wt % based on total weight of the tablet, excluding any coating):
palonosetron hydrochloride in an amount of about 0.1 to about 2.0 wt %, preferably about 0.15 to about 0.25 wt %, and more preferably about 0.18 to about 0.25 wt %;
diluent in an amount of about 60 to about 80 wt %, preferably about 65 to about 75 wt % and most preferably about 68 to about 72 wt %, preferably wherein the diluent is a mixture of lactose and microcrystalline cellulose;
binder in an amount of about 1 to about 8 wt %, preferably about 2 to about 6 wt %, and preferably wherein the binder is povidone;
disintegrant in an amount of about 15 to about 30 wt %, preferably about 20 to about 28 wt % and more preferably about 22 to about 26 wt %, and preferably wherein the disintegrant contains crospovidone and a mixture of sodium bicarbonate and tartaric acid; and
lubricant in an amount of about 0.5 to about 1.5 wt %, preferably about 1 to about 1.2 wt %, and preferably wherein the lubricant is magnesium stearate.

The present invention further provides a process for the preparation of the dosage forms by dry or wet granulation, preferably by dry granulation, of palonosetron or a pharmaceutically acceptable salt thereof and at least one pharmaceutically acceptable excipient. The dry granulation is preferably conducted in a low shear mixer.

The present invention further provides a blend obtainable by the processes described herein. The blend can be incorporated in the dosage forms of the present invention, e.g. as a capsule filling, or as a blend for compressing into tablets.

The dosage forms and the blends of any embodiment of the present invention preferably have a blend uniformity of about 90% to about 110% (or of about 95% to about 105%), with an associated relative standard deviation (RSD) of about 5% or less, preferably about 4% or less, more preferably about 3% or less, more preferably about 2% or less, most preferably about 1% or less and particularly about 0.7% or less, as measured by the concentration of palonosetron or pharmaceutically acceptable salt thereof. Further, the dosage forms of any embodiment of the present invention have a content uniformity of about 90% to about 110% (or of about 95% to about 105%) with RSD of about 5% or less, preferably about 4% or less, more preferably about 3% or less, more preferably about 2% or less, and most preferably about 1% or less, and particularly about 0.7% or less, as measured by the concentration of palonosetron or pharmaceutically acceptable salt thereof. Preferably, the blend and content uniformity is determined using 10 samples, each sample being equivalent to the approximate weight of one capsule fill (i.e. about 50 mg to about 400 mg, preferably about 100 to about 300 mg, more preferably about 150 to about 300 mg and most preferably about 200 mg to about 300 mg,). Typically, the sample weight is about 200 mg. Determination of the palonosetron or pharmaceutically acceptable salt content may be carried out by any suitable procedure, such as HPLC using UV detection.

The dosage forms of any embodiment of the present invention preferably have a dissolution profile such that greater than about 70%, more preferably greater than about 80%, most preferably greater than about 90%, and particularly greater than about 95%, or greater than about 98% by weight of the dosage form dissolves in 15 minutes when measured using the USP paddle method at 75 rpm in 500 ml of 0.01N HCl at pH 2 at 37° C.

Further, the dosage forms and blends of any embodiment of the present invention are stable, preferably such that about 1% or less, preferably about 0.5% or less, more preferably about 0.1% or less, and most preferably about 0.05% or less palonosetron N-oxide is present after storage for 90 days at 40° C. and 75% relative humidity.

Alternatively, the dosage forms and blends of any embodiment of the present invention are stable, preferably such that any increase in the amount of palonosetron N-oxide (a known degradant disclosed in the prior art such as US2008/0152704) after storage of the dosage forms and blends for 90 days at 40° C. and 75% relative humidity is about 1% or less, preferably about 0.5% or less, more preferably about 0.1% or less, and most preferably about 0.05% or less, wherein the % is wt % or HPLC area % based on the initial weight or HPLC area of the palonosetron in the dosage form or blend at the start of the storage. Most preferably, the dosage form or blend of any embodiment of the present invention is stable such at there is about 0% increase in palonosetron N-oxide after the storage.

The dosage forms and blends of any embodiment of the present invention may be stable such that about 1% or less, preferably about 0.5% or less, or preferably about 0.1% or less, and most preferably about 0.05% or less total degradant is present after storage for 90 days at 40° C. and 75% relative humidity.

The dosage forms and blends of any embodiment of the present invention may be stable such that any increase in the amount of total degradant(s) of palonosetron after storage of the dosage forms and blends for 90 days at 40° C. and 75% relative humidity is about 1% or less, preferably about 0.5% or less, or preferably about 0.1% or less, and most preferably about 0.05% or less, wherein the % is wt % or HPLC area % based on the initial weight or HPLC area of the palonosetron in the dosage form or blend at the start of the storage. Most preferably, the dosage form or blend of any embodiment of the present invention is stable such at there is about 0% increase in the amount of the total degradant(s) or any unknown impurity/ies after the storage.

More preferably, the dosage forms and blends of any embodiment of the present invention may be stable such that after storage for 90 days at 40° C. and 75% relative humidity, the relative assay is about 97% or more (e.g. about 97% to about 101%, preferably about 97% to about 100%), preferably about 98% or more (e.g. about 98% to about 101%, preferably about 98% to about 100%), more preferably about 99% or more (e.g. about 99% to about 101%, preferably about 99% to about 100%), and most preferably about 99.5% or more (e.g. about 99.5% to about 101%, preferably about 99.5% to about 100%) of the palonosetron in the dosage form or blend at the start of the storage.

The invention further provides a dosage form obtainable by any of the processes described herein.

### DETAILED DESCRIPTION OF THE INVENTION

As used herein, and unless otherwise indicated, the term palonosetron is taken to include pharmaceutically acceptable salts thereof. Preferably the palonosetron in the formulations and processes of the present invention is in the form of palonosetron hydrochloride.

As used herein, the term “lactose” includes all forms of lactose, including spray-dried lactose, lactose monohydrate, anhydrous lactose or amorphous lactose or mixtures thereof. Preferably in the formulations and processes of the present invention, the lactose is in the form of lactose monohydrate, or mixtures of lactose monohydrate and amorphous lactose. Particularly preferred is spray dried lactose (such as lactose DCL 14), lactose 100 mesh and lactose 200 mesh. Preferably, lactose 100 mesh has a particle size distribution range: <63 μm: 0-15%, <150 μm: 60-80% and <250 μm: 99-100%. Preferably lactose 200 mesh has a particle size distribution range: <45 μm: 50-65%, <100 μm: 90-100%, <150 μm: 96-100% and <250 μm: 99-100%. Preferably spray dried lactose has a particle size distribution range of: <45 μm: 0-15%, <100 μm: 30-60%, and <250 μm: 98-100%.

As used herein, and unless otherwise indicated, percentages of excipients are given as weight % relative to the weight of the dosage form excluding any capsule shell or tablet coating where present.

The terms “high shear” and “low shear” in relation to mixers are well known in the field of pharmaceutics. Typically, low shear mixers, such as tumble type mixers, flow bin mixers, twin shell mixers and Y cone mixers, have rotary or vibratory paddles or fins. Y cone mixers are preferred for the low shear processes of the present invention. Typically, high shear mixers, such as plough-share mixers (e.g. Fielder Mixer and Diosna® mixers) have a rotor and stator construction; Diosna® mixers are preferred for the high shear processes of the present invention.

The present invention provides a dosage form for oral administration comprising a solid admixture of palonosetron or a pharmaceutically acceptable salt thereof and at least one pharmaceutically acceptable excipient. The dosage form for oral administration of the present invention can be a solid dosage form. The dosage form may be in the form of a powder (e.g. as a sprinkle formulation) or granules. The powder or granules may be encapsulated, i.e. the dosage form may be in the form of a filled hard gelatin capsule or formed into a tablet (e.g. by compression). The dosage form may also be in the form of an effervescent dosage form.

In a preferred embodiment, the dosage form of the present invention is a hard gelatin capsule, which comprises a gelatin outer shell and a solid admixture of palonosetron or a pharmaceutically acceptable salt thereof with at least one pharmaceutically acceptable excipient. The admixture of palonosetron or a pharmaceutically acceptable salt thereof with at least one pharmaceutically acceptable excipient is in the solid phase, preferably in the form of a granule or a powder.

The dosage forms of the present invention preferably provides palonosetron having a bioavailability that is comparable with the palonosetron bioavailability of a liquid capsule formulation such as the liquid capsule formulation, Aloxi® capsules containing palonosetron hydrochloride, and the following excipients: monoglycerides and diglycerides of capryl/capric acid, glycerin, polyglyceryl oleate, water and butylated hydroxyanisole, approved 22 Aug. 2008 under FDA Application No. (NDA) 02223.

The dosage forms of any of the embodiments of the present invention preferably have a favourable dissolution profile. In particular, greater than about 70%, more preferably greater than about 80%, most preferably greater than about 90%, and particularly greater than about 95%, or greater than about 98% by weight of the dosage form dissolves in 15 minutes when measured using the USP paddle method at 75 rpm in 500 ml of 0.01N HCl at pH 2 at 37° C.

Preferably the dosage forms of any embodiment of the present invention have a good blend and/or content uniformity. In particular, the solid admixture in the dosage form has a blend uniformity of preferably about 90% to about 110% (or between about 95% and about 105%), with a relative standard deviation (RSD) of about 5% or less, preferably about 3% or less. Preferably, the solid admixture has a blend uniformity of about 90% to about 110% with RSD of less than about 3%. Furthermore, the dosage forms of any embodiment of the present invention preferably have a content uniformity between about 90% and 110% with RSD of about 5% or less. Preferably, the content uniformity is between about 90% and about 110% with RSD less than about 3% as measured by the concentration of palonosetron or pharmaceutically acceptable salt thereof.

The dosage forms of the present invention are stable. Preferably, the dosage forms of the present invention have a greater stability compared with aqueous injection formulations. Furthermore, the dosage forms of the present invention are advantageously easy to manufacture, and are stable without the need to incorporate any antioxidant excipients such as butylated hydroxyanisole, or reducing agents, and without the need to include chelating agents such as ethylenediamine tetraacetic acid. Furthermore, the solid dosage forms of the present invention preferably do not require the use of e.g. gelatin capsule shells having a particular oxygen permeability in order to ensure storage stability. Conveniently, the dosage forms of the present invention may employ any suitable hard gelatin capsule typically used for pharmaceutical dosage forms. Nevertheless, an antioxidant excipient may be included to further enhance the stability of the dosage form, especially in oral dispersible tablets or effervescent disintegrant systems.

In a preferred embodiment, after storage for 3 months at 40° C. and 75% relative humidity, no more than about 10%, preferably no more than about 5%, and more preferably no more than about 1% by weight, further preferably 0.5% or less, and most preferably 0.1% or less of the labelled amount of palonosetron (the initially present amount in the dosage form) degrades into palonosetron N-oxide Alternatively, in preferred embodiments of the present invention, about 2% or less, preferably about 1% or less, more preferably about 0.5% or less and most preferably about 0.2% or less, or about 0.1% or less by weight of the initially present amount of palonosetron in the dosage form degrades into palonosetron N-oxide after 5 days at 55° C., 100% relative humidity.

In another preferred embodiment, the pharmaceutical composition of the present invention has not more than about 5%, preferably not more than about 2%, more preferably not more than about 1% reduction in assay when tested after storage for 2 months at 40° C. and 75% relative humidity.

The dosage form of the present invention is particularly suitable for providing a amount of about 0.02 mg to about 10 mg, preferably about 0.05 mg to about 5 mg, more preferably about 0.1 mg to about 2 mg, and most preferably about 0.1 mg to about 5 mg per dosage form based on the weight of palonosetron base. Typically, the dosage form provides about 0.25 mg, 0.5 mg or 0.75 mg based on the weight of palonosetron base.

The palonosetron or pharmaceutically acceptable salt thereof in any of the dosage forms of the present invention may be present in a concentration of about 0.05 to about 5 wt %, preferably about 0.1 to about 2 wt %, more preferably about 0.1 to about 0.5 wt %, and most preferably about 0.2 to about 0.35 wt % or about 0.25 to about 0.30 wt %, wherein the wt % is relative to the weight of the dosage form excluding any tablet coating or capsule shell.

Palonosetron in the form of its hydrochloride salt is the preferred in the dosage forms of the present invention.

As mentioned above, the dosage form of the present invention can be in any form provided that the drug is present in a solid admixture with at least one pharmaceutically acceptable excipient. For example, the dosage may be in the form of a powder, where the powder can be encapsulated (e.g. in a hard gelatin capsule) or in the form of a sprinkle formulation. In other embodiments, the dosage form of the present invention can be in the form of a tablet, including orally disintegrating tablet, or effervescent tablet, wherein the tablets may be compressed, and wherein the tablets may optionally be coated. For example, the dosage form of the present invention can be in the form of a filled hard gelatin capsule, comprising a gelatin outer shell, and a solid admixture of palonosetron or a pharmaceutically acceptable salt thereof, preferably palonosetron hydrochloride, with at least one pharmaceutically acceptable excipient.

The capsule shell of the preferred dosage form of the present invention is preferably a hard gelatin capsule of the type commonly used for pharmaceutical dosage forms. Such capsules typically comprise gelatin and plasticizers, and may optionally contain excipients such as preservatives, colours, opacifying agents and flavours.

Suitable plasticizers include glycerin, sorbitol, dextrin, glycerin, mannitol, palmitic acid, and polyethylene glycol.

Preservatives in the capsule shell, if included, can include parabens (e.g. methylparaben, ethylparaben and propylparaben).

Particularly preferred capsules are two part hard gelatin capsules composed of gelatin, opacifying agent (e.g. titanium dioxide), and colours (e.g. iron oxides). Since the solid admixture of palonosetron or pharmaceutically acceptable salts thereof is stable, the dosage forms of any embodiment of the present invention do not require the presence of an antioxidant. However, the stability can be further enhanced by the use of an antioxidant excipient. Further, the use of capsules having a low oxygen permeability is also unnecessary.

Since the active agent is present in a solid admixture with excipients, the dosage forms of the present invention offer a further advantage in their ease of handling and manufacture compared to liquid soft capsule formulations and i.v. solutions.

The dosage forms of any embodiment of the present invention preferably includes at least one pharmaceutically acceptable excipient selected from the group consisting of a binder, disintegrant, diluent and lubricant. A dosage form according to the invention can include a pharmaceutically acceptable excipient, wherein the pharmaceutically acceptable excipient is a combination of at least one binder, at least one disintegrant, at least one diluent, and at least one lubricant, and optionally at least one glidant. A glidant is preferably used when the dosage form is a tablet such as an orally disintegrating tablet. The glidant is preferably colloidal silicon dioxide.

Preferred binders include gelatin, cellulose, cellulose derivatives (e.g. hydroxypropyl cellulose, hydroxypropyl methyl cellulose), polyvinylpyrrolidone (povidone), starch, sucrose and polyethylene glycol xylitol, sorbitol, maltitol. Povidone (e.g. PVP K30) is especially preferred, particularly in the filled hard gelatin capsule embodiments of the present invention. Starch is particularly preferred if the composition is to be manufactured by wet granulation. Also preferred are dosage forms according to the present invention wherein the binder is povidone. Where povidone is used, the povidone preferably has a K-value of about 27 to about 32, preferably about 29 to about 31, and preferably about 30 (such as povidone K30).

In particularly preferred dosage forms of any embodiment of the present invention, the binder contains lactose, either alone, or in combination with calcium hydrogen phosphate (particularly anhydrous calcium hydrogen phosphate), or with mannitol. The binder may also consist of a combination of mannitol with calcium hydrogen phosphate (particularly anhydrous calcium hydrogen phosphate).

The binder is preferably present in an amount of about 1 to about 10 wt %, preferably about 2 to about 8 wt %, and more preferably about 3 to about 6 wt % or 3 to about 5 wt %, and most preferably about 5 wt % based on the weight of the dosage form excluding any capsule shell or tablet coating.

Particularly useful disintegrants include alginic acid, carboxymethylcellulose sodium or calcium, cellulose, colloidal silicon dioxide, croscarmellose sodium, crospovidone, hydroxypropyl cellulose, magnesium aluminium silicate, microcrystalline cellulose, sodium bicarbonate, sodium starch glycolate, pregelatinized starch, tartaric acid, citric acid and mixtures thereof, and preferably wherein the disintegrant is selected from pregelatinized starch, carboxymethylcellulose sodium or calcium, cellulose, croscarmellose sodium, crospovidone, and sodium starch glycolate. Preferably, the disintegrant is selected from the group consisting of alginic acid, carboxymethylcellulose sodium or calcium, cellulose, colloidal silicon dioxide, croscarmellose sodium, crospovidone, magnesium aluminium silicate, microcrystalline cellulose, sodium starch glycolate, starch, and mixtures thereof. Most preferably the disintegrant is selected from the group consisting of pregelatinized starch, croscarmellose sodium, and sodium starch glycolate. Especially preferred are superdisintegrants such as croscarmellose sodium (e.g. Ac-Di-Sol®, an internally crosslinked sodium carboxymethylcellulose), sodium starch glycolate and crospovidone or mixtures thereof, more preferably croscarmellose sodium (Ac-Di-Sol®) and sodium starch glycolate or mixtures thereof, and most preferably croscarmellose sodium (Ac-Di-Sol®). For orally disintegrating tablet or effervescent formulations, the disintegrant component can also include an effervescent disintegrant system whereby two components are used, which in the presence of moisture, generates a gas such as carbon dioxide to provide rapid dissolution or disintegration of the drug. For example, an alkali metal or alkaline earth metal bicarbonate or carbonate, and a pharmaceutically acceptable organic acid. Preferred alkali metal and alkaline earth metal bicarbonates and carbonates are sodium carbonate, potassium carbonate, magnesium carbonate, calcium carbonate, sodium bicarbonate and potassium bicarbonate. Sodium bicarbonate is more preferred. Preferred pharmaceutically acceptable organic acids include citric acid and/or tartaric acid, with tartaric acid being particularly preferred. An especially preferred effervescent disintegrant system comprises sodium bicarbonate citric acid and tartaric acid, more preferably sodium bicarbonate and tartaric acid. For orally disintegrating tablets, the disintegrant may include croscarmellose sodium.

In any embodiment of the present invention, the disintegrant is preferably selected from the group consisting of pregelatinized starch, croscarmellose sodium, or a mixture or croscarmellose sodium with an effervescent disintegrant system, or a mixture of croscarmellose sodium with pregelatinized starch, or a mixture of sodium starch glycolate, pregelatinized starch and croscarmellose sodium. Also preferred are formulations according to any embodiment of the present invention wherein the disintegrant consists of sodium starch glycolate. In a particularly preferred embodiment, the effervescent disintegrant combination of sodium bicarbonate and tartaric acid is used in combination with the crospovidone in orally disintegrating tablet formulations or effervescent formulations of the present invention.

When the dosage form is an orally disintegrating tablet or effervescent tablet, the disintegrant is preferably present in an amount of about 5 to about 30 wt %, more preferably about 10 to about 25 wt % and most preferably about 12 to about 20 wt % of the dosage form excluding any tablet coating.

In preferred capsule formulations of the present invention, the disintegrant is preferably present in an amount of about 5 to about 15 wt %, particularly about 8 to about 12 wt %, and more particularly about 10 wt % of the dosage form, excluding any capsule shell or tablet coating.

Preferably when the dosage form is in the form of a filled hard gelatin capsule the disintegrant is croscarmellose sodium or crospovidone, preferably croscarmellose sodium, and may be present in an amount of about 8 to about 20 wt %, more preferably about 10 to about 12 wt % and most preferably about 10 wt % of the dosage form, excluding the capsule shell.

When the dosage form of is in the form of an orally disintegrating tablet or effervescent tablet, and particularly an effervescent tablet, the disintegrant can comprise crospovidone and additionally an effervescent disintegrant (such as sodium bicarbonate and an organic acid, such as citric acid or tartaric acid). Preferably the effervescent disintegrant system is sodium bicarbonate and tartaric acid. Typically the sodium bicarbonate and tartaric acid is present in a combined amount of about 2 to about 20 wt %, more preferably about 5 to about 13 wt % and most preferably about 6 to about 10 wt %, and especially about 8 wt % of the dosage form, excluding any tablet coating.

Suitable diluents for any of the dosage forms of the present invention can be selected from the group consisting of calcium carbonate, calcium hydrogen phosphate (also known as dicalcium phosphate, calcium monohydrogen phosphate or dibasic calcium phosphate) (preferably the calcium hydrogen phosphate is in anhydrous form), cellulose, microcrystalline cellulose, ethylcellulose, magnesium carbonate, magnesium oxide, mannitol, dextrin, dextrose, sorbitol, lactose, starch, sucrose, talc, tragacanth, xylitol, and mixtures thereof. Preferred diluents are lactose, microcrystalline cellulose, and mannitol, starch and calcium hydrogen phosphate (preferably anhydrous calcium hydrogen phosphate), with lactose and microcrystalline cellulose or a combination thereof being particularly preferred. Combinations of mannitol and calcium hydrogen phosphate, or mannitol and lactose may also be used in the dosage forms of any embodiment of the present invention. Preferably, when calcium hydrogen phosphate (more preferably anhydrous calcium hydrogen phosphate) is used, it is present in an amount of less than about 70 wt %, preferably less than about 50 wt %, preferably 40% or less and more preferably in an amount of about 30 to about 40 wt %, excluding any capsule shell or tablet coating. The calcium hydrogen phosphate (and preferably anhydrous calcium hydrogen phosphate) may be used in combination with another diluent, such as lactose.

An especially preferred diluent for any of the dosage forms of the present invention is lactose; most preferably the diluent is lactose monohydrate. The lactose preferably comprises crystalline alpha lactose monohydrate or amorphous lactose or mixtures thereof. Particularly suitable is lactose 100 mesh, lactose 200 mesh, or spray dried lactose (mixture of alpha lactose monohydrate and amorphous lactose for direct compression) as described above, or mixtures thereof, preferably wherein the lactose is a mixture of lactose 100 mesh and spray dried lactose, or wherein the lactose is a mixture of lactose 100 mesh, lactose 200 mesh and spray dried lactose for direct compression.

The diluent is preferably present in an amount of about 30 to about 90 wt %, preferably about 50 to about 85 wt %, most preferably about 65 to about 85 wt % or about 65 to about 80 wt %, or preferably about 75 to about 85 wt % or about 65 to about 80 wt %, excluding any capsule shell or tablet coating.

The dosage forms of any embodiment of the present invention preferably include a lubricant. Suitable lubricants include those selected from consisting of calcium stearate, glycerin monostearate, magnesium lauryl sulfate, magnesium stearate, sodium benzoate, sodium lauryl sulfate, sodium stearyl fumarate, stearic acid, talc and zinc stearate or mixtures thereof. Sodium stearyl fumarate or magnesium stearate, or mixtures thereof are particularly preferred, especially for the filled hard capsule dosage forms of the present invention. Sodium stearyl fumarate is an especially preferred lubricant.

Preferably the lubricant is present in an amount of about 0.1 to about 2 wt %, preferably about 0.5 to about 1.2 wt %, or about 0.5 to about 1.5 wt % and most preferably about 0.8 to about 1.5 wt % excluding any capsule shell or tablet coating.

In other embodiments of the present invention, the lubricant is present in an amount of about 0.5 to about 2.5 wt %, preferably about 1 to about 2.5 wt %, more preferably about 1.4 to about 2.1 wt %, and most preferably about 1.5 wt %, excluding any capsule shell or tablet coating.

In any embodiment of the invention, sodium stearyl fumarate is a preferred lubricant and is preferably used in an amount of about 0.5 to about 6 wt %, preferably about 1 to about 5 wt % and more preferably about 1 to about 3 wt %, or about 1 to about 2 wt %, particularly about 1.5 wt % based on the dosage form excluding capsule shell or coating. Magnesium stearate may be used preferably in an amount of about 0.25 to about 2 wt %, preferably about 0.25 to about 1 wt %, and particular about 0.5 wt % based on the dosage form excluding capsule shell or coating.

The dosage forms of the present invention, particularly in the tablet (such as orally disintegrating tablet) formulations, may further comprise a glidant. The glidant is preferably colloidal silicon dioxide. The glidant may be present in an amount of about 0.1 to about 0.5 wt %, preferably 0.1 to about 0.3 wt %, more preferably about 0.2 to about 0.3 wt % relative to the weight of the dosage for excluding any capsule shell or coating. Colloidal silicon dioxide in an amount of about 0.1 to about 0.5 wt %, more preferably about 0.2 to about 0.3 wt % of the dosage form, is a particularly preferred glidant. Preferably, these formulations contain diluent which is preferably lactose or mannitol, or a mixture thereof (preferably a mixture thereof), preferably wherein the diluent is present in an amount of about 75 to about 85 wt %, more preferably about 80 to about 85 wt % of the dosage form. Croscarmellose sodium and/or pregelatinized starch, or a mixture thereof, are especially preferred disintegrants for this embodiment. The disintegrant is preferably present in an amount of about 8 to about 25 wt %, more preferably about 10 to about 20 wt % of the dosage form. The orally disintegrating tablets of the present invention may further comprise a flavouring and/or a sweetener (typically in amounts of about 1 to about 4 wt %, preferably about 2 to about 3 wt %). Such dosage forms may be made by a wet or dry granulation process, preferably by a wet granulation process.

For the dosage forms of the present invention such as sprinkle formulations, powders or orally disintegrating tablets, these may further comprise flavours, sweeteners and taste masking agents or a combination thereof.

The present invention further provides a dosage form in the form of a filled hard gelatin capsule wherein the disintegrant is crospovidone, preferably wherein the crospovidone is present in an amount of about 10 to about 20 wt %, more preferably about 12 to about 18 wt % and most preferably about 16 wt % of the dosage form, excluding the capsule shell.

In a further embodiment, the present invention provides a dosage form in the form of an orally disintegrating tablet or effervescent tablet wherein the disintegrant is selected from crospovidone, and an effervescent disintegrant, or a mixture thereof, and is preferably a mixture of crospovidone and an effervescent disintegrant. The crospovidone is preferably present in an amount of about 10 to about 25 wt %, preferably about 15 to about 25 wt %, and more preferably about 16 wt % of the dosage form, excluding any coating. The effervescent disintegrant is preferably a mixture of sodium bicarbonate and tartaric acid, preferably in a wt/wt ratio of about 2:1 to about 1:2, preferably about 1.5:1 to about 1:1.5 and preferably about 1:1. The sodium bicarbonate and tartaric acid is typically present in a combined amount of about 2 to about 20 wt %, more preferably about 5 to about 13 wt % and most preferably about 6 to about 10 wt %, and especially about 8 wt % of the dosage form, excluding any tablet coating.

Particularly preferred dosage forms of the present invention include the following:

1. A hard gelatin capsule dosage form consisting of (wt % based on total weight of the capsule filling, and excluding the capsule shell): palonosetron hydrochloride in an amount of about 0.1 to about 2.0 wt %; diluent in an amount of about 60 to about 85 wt %; binder in an amount of about 1 to about 8 wt %; disintegrant in an amount of about 5 to about 25 wt %; preferably about 10 to about 25 wt %; and lubricant in an amount of about 0.5 to about 2 wt %, preferably about 0.5 to about 1.5 wt %;

2. A hard gelatin capsule dosage form consisting of (wt % based on total weight of the capsule filling, and excluding the capsule shell): palonosetron hydrochloride in an amount of about 0.2 to about 0.35 wt %, preferably about 0.25 to about 0.3 wt % and more preferably about 0.28 wt %; diluent in an amount of about 75 to about 85 wt %, preferably about 80 to about 85 wt %, and more preferably about 83 wt %; binder in an amount of about 3 to about 6 wt %, preferably about 4 to about 6 wt %, and more preferably about 5 wt %; disintegrant in an amount of about 5 to about 15 wt %, preferably about 8 to about 12 wt %, and more preferably about 10 wt %; and lubricant in an amount of about 1 to about 2.5 wt %, preferably about 1.1 to about 2.4 wt %, and more preferably about 1.5 wt %;

3. A hard gelatin capsule according to any of the above described embodiments wherein: the diluent is selected from the group consisting of: lactose, microcrystalline cellulose, mannitol, starch or calcium hydrogen phosphate (preferably anhydrous calcium hydrogen phosphate), and mixtures thereof; the binder is selected from the group consisting of: gelatin, cellulose, cellulose derivatives, polyvinylpyrrolidone (povidone), starch, sucrose and polyethylene glycol xylitol, sorbitol, maltitol, and mixtures thereof; the disintegrant is selected from the group consisting of alginic acid, carboxymethylcellulose sodium or calcium, cellulose, colloidal silicon dioxide, croscarmellose sodium, crospovidone, hydroxypropyl cellulose, magnesium aluminium silicate, microcrystalline cellulose, sodium bicarbonate, sodium starch glycolate, starch, and mixtures thereof; and the lubricant is selected from the group consisting of: calcium stearate, glycerin monostearate, magnesium lauryl sulfate, magnesium stearate, sodium benzoate, sodium lauryl sulfate, sodium stearyl fumarate, stearic acid, talc and zinc stearate, and mixtures thereof;

4. A hard gelatin capsule according to any of the above-described embodiments wherein: the diluent is selected from the group consisting of lactose, microcrystalline cellulose, mannitol and starch, and mixtures thereof; the binder is selected from the group consisting of polyvinylpyrrolidone (povidone), starch polyethylene glycol, and mixtures thereof; the disintegrant is selected from the group consisting of carboxymethylcellulose sodium or calcium, colloidal silicon dioxide, croscarmellose sodium, crospovidone, sodium starch glycolate, and mixtures thereof; and the lubricant is selected from the group consisting of calcium stearate, glycerin monostearate, magnesium stearate, sodium lauryl sulfate, sodium stearyl fumarate, stearic acid, talc and zinc stearate, and mixtures thereof;

5. A hard gelatin capsule according to any of the above-described embodiments wherein: the diluent is selected from the group consisting of: lactose and starch, or mixtures thereof; the binder is polyvinylpyrrolidone (povidone); the disintegrant is selected from the group consisting of croscarmellose sodium or sodium starch glycolate, and mixtures thereof; and the lubricant is selected from the group consisting of magnesium stearate or sodium stearyl fumarate and mixtures thereof.

6. A dosage form according to any of the above-described embodiments wherein: the diluent is lactose, preferably comprising alpha lactose monohydrate, more preferably wherein the lactose is selected from lactose 100 mesh, lactose 200 mesh or spray dried lactose (mixture of alpha lactose monohydrate and amorphous lactose for direct compression) or mixtures thereof, preferably wherein the lactose is a mixture of lactose 100 mesh and spray dried lactose, or wherein the lactose is a mixture of lactose 100 mesh, lactose 200 mesh and spray dried lactose for direct compression; the binder is polyvinylpyrrolidone (povidone), preferably wherein povidone K30; the disintegrant is croscarmellose sodium; and the lubricant is sodium stearyl fumarate.

The invention further provides an orally disintegrating tablet or effervescent dosage form consisting of (wt % based on total weight of the tablet, excluding any coating): palonosetron hydrochloride in an amount of about 0.1 to about 2.0 wt %, preferably about 0.15 to about 0.25 wt %, and more preferably about 0.18 to about 0.25 wt %; diluent in an amount of about 60 to about 80 wt %, preferably about 65 to about 75 wt % and most preferably about 68 to about 72 wt %, preferably wherein the diluent is a mixture of lactose and microcrystalline cellulose; binder in an amount of about 1 to about 8 wt %, preferably about 2 to about 6 wt %, and preferably wherein the binder is povidone; disintegrant in an amount of about 15 to about 30 wt %, preferably about 20 to about 28 wt % and more preferably about 22 to about 26 wt %, and preferably wherein the disintegrant contains crospovidone and a mixture of sodium bicarbonate and tartaric acid; and lubricant in an amount of about 0.5 to about 1.5 wt %, preferably about 1 to about 1.2 wt %, and preferably wherein the lubricant is magnesium stearate.

In another embodiment of the present invention, the orally disintegrating tablet of the present invention consists of (wt % based on total weight of the tablet, excluding any coating): palonosetron hydrochloride in an amount of about 0.1 to about 2.0 wt %, preferably about 0.15 to about 0.25 wt %, and more preferably about 0.18 to about 0.25 wt %; diluent in an amount of about 75 to 85 wt %, preferably wherein the diluent is a mixture of lactose and mannitol; binder in an amount of about 2 to about 6 wt %, preferably about 3 to about 5 wt %, and preferably wherein the binder is pregelatinized starch; disintegrant in an amount of about 5 to about 20 wt %, preferably about 8 to about 12 wt %, preferably wherein the disintegrant is croscarmellose sodium; and lubricant in an amount of about 1 to about 2.0 wt %, preferably about 1.2 to about 1.7 wt %, and preferably wherein the lubricant is sodium stearyl fumarate.

Preferably, the dosage forms of any embodiment of the present invention does not contain an antioxidant.

Preferably, the dosage forms of any embodiment of the present invention have a content uniformity between 90-110% with RSD of less than 5%, preferably a content uniformity of 90-110% with RSD less than 3%, and are preferably filled hard gelatin capsules. Preferably the dosage forms are prepared by dry granulation in a low shear mixer, such as a Y-cone mixer, a tumble type mixer, flow bin mixer or a twin shell mixer, preferably a Y-cone mixer.

The dosage forms of the present invention have a dissolution profile such greater than 70%, more preferably greater 80%, most preferably greater than 90%, and particularly greater than 95%, or greater than 98% by weight of the dosage form dissolves in 15 minutes when measured using the USP paddle method at 75 rpm in 500 ml of 0.01N HCl at pH 2 at 37° C.

The dosage forms of the present invention preferably have a stability such that 1% or less, preferably 0.5% or less, or preferably 0.1% or less palonosetron N-oxide is present after storage for 90 days at 40° C. and 75% relative humidity. The stability is achieved without the use of any antioxidant excipients, and without the need to provide an oxygen impermeable barrier such as capsules having a low oxygen permeability or oxygen-impermeable coating.

The present inventors have surprisingly found that although the drug is present in a solid phase in the dosage forms of the present invention, rather than a liquid phase, the dosage forms of any embodiment of the present invention are capable of providing palonosetron or a pharmaceutically acceptable salt thereof having a bioavailability that is comparable to the palonosetron bioavailability of a liquid soft gelatin capsule formulation. In particular, the dosage forms of the present invention have a bioavailability that compares favourably with Aloxi® capsules containing palonosetron hydrochloride, and the following excipients: monoglycerides and diglycerides of capryl/capric acid, glycerin, polyglyceryl oleate, water and butylated hydroxyanisole [approved 22 Aug. 2008 under FDA Application No. (NDA) 02223]. Typically the formulations of the present invention may have bioequivalence, compared with Aloxi® capsules, of about 80% to about 125% as measured by AUC, and of about 80% to about 125% as measured by Cₘₐₓ or an average maximum plasma concentration, in comparison with Aloxi® capsules.

Preferably, the dosage forms of any embodiment of the present invention provides a dissolution profile of about 60 to 90%, more preferably about 70 to about 85%, and especially about 80% in 15 minutes, when measured USP paddle method of 75 rpm in 500 ml 0.01N HCl at pH 2 at 37° C. Preferably, no less than about 70%, more preferably no less than about 80% of the palonosetron in the dosage form is dissolves in 15 minutes. Most preferably at least 90% dissolves in 15 minutes. In especially preferred embodiments, 95% or more dissolves in 15 minutes.

The formulations of the present invention are stable. It is known that prior art liquid formulations of palonosetron suffer from stability problems. In particular, aqueous injection formulations are unstable and suffer from storage problems. The prior art liquid soft gel capsules apparently also suffer from stability problems, and require the presence of an antioxidant such as butylated hydroxyanisole to maintain storage stability. In particular, the formulations of the present invention including the hard gelatin capsules of the present invention, are especially stable without the need to include antioxidant excipients. Thus, preferably the formulations of the present invention do not contain antioxidants such as butylated hydroxyanisole, butylated hydroxytoluene and reducing agents. More preferably the formulations of the present invention do not contain butylated hydroxyanisole.

In preferred embodiments of the present invention, the dosage form is in the form of a powder or granules (such as a sprinkle formulation). Preferably, the powder or granules are encapsulated in a hard gelatin capsule. The weight of the dosage form (excluding the capsule shell is preferably in the range of about 50 mg to about 400 mg, preferably about 100 to about 300 mg, more preferably about 150 to about 300 mg and most preferably about 200 mg to about 300 mg.

The present invention also encompasses a process for the preparation of the any of the dosage forms of the invention.

The dosage forms of the present invention can be made by direct compression, dry or wet granulation. Dry granulation or direct compression is preferred, and dry granulation is particularly preferred. Dry granulation is preferably conducted in low shear mixer.

Solid pharmaceutical composition of Palonosetron is preferably prepared by conventional dry granulation. Dry granulation can be performed, for example, by compaction or slugging. Compaction techniques are well known in the art and typically include the use of roller compactor. Slugging is a common technique in the field and involves the use of a tableting machine to produce slugs and passing the slugs through a mill or an oscillating granulator to form granules. The granules may subsequently be milled and filled into hard gelatin capsule or be compressed into a tablet.

As an alternative to dry granulation, a blended composition may be compressed directly into a compacted dosage form using direct compression techniques.

When using dry granulation or direct compression, the mixing of ingredients is preferably done in a geometric dilution process, for example first mixing the active drug with a smaller quantity of excipient and then building up the volume to make sure the active ingredient is properly distributed.

In the dry granulation process, the method comprises:

(1) providing a mixture of palonosetron or a pharmaceutically acceptable salt thereof, and at least one pharmaceutically acceptable excipient selected from the group consisting of: diluent, binder, disintegrant, and combination thereof;

(2) blending to obtain a first mixture;

(3) admixing one or more excipients to the first mixture to form a second mixture;

(4) compressing the second mixture;

(5) milling the compressed mixture; and

(6) blending the milled mixture with one or more excipients.

The mixture from step (6) may optionally be filled into capsules to provide a hard gelatin capsule formulation, or compressed into a tablet to provide a tablet formulation, and optionally coating the tablet.

The mixture in step (1) preferably comprises palonosetron or a pharmaceutically acceptable salt thereof, at least one diluent, at least one binder and at least one disintegrant, and preferably includes a diluent, binder and disintegrant. The palonosetron or pharmaceutically acceptable salt thereof; at least one diluent, at least one binder and at least one disintegrant are added stepwise, for example, the palonosetron or pharmaceutically acceptable salt thereof, binder and disintegrant are added stepwise, each in admixture with a portion of the diluent (geometric dilution). Suitable binders, diluents and disintegrants are described above. The process is particularly suitable for the filled gelatin capsule dosage forms of the present invention.

In the above process, the mixture in step (1) preferably comprises palonosetron or a pharmaceutically acceptable salt thereof and at least one diluent. The diluent is preferably selected from the group consisting of calcium carbonate, calcium hydrogen phosphate (preferably anhydrous calcium hydrogen phosphate), cellulose, microcrystalline cellulose, ethylcellulose, magnesium carbonate, magnesium oxide, mannitol, dextrin, dextrose, sorbitol, lactose, starch, sucrose, talc, tragacanth, xylitol, and mixtures thereof. More preferably, the diluent is selected from the group consisting of lactose, microcrystalline cellulose and mixtures thereof. More preferably, the diluent is lactose. Preferably the disintegrant is selected from the group consisting of alginic acid, calcium phosphate tribasic, carboxymethylcellulose sodium or calcium, cellulose, colloidal silicon dioxide, croscarmellose sodium, crospovidone, hydroxypropyl cellulose, magnesium aluminium silicate, microcrystalline cellulose, sodium starch glycolate, starch, and mixtures thereof. The preferred disintegrant in step (1) is crospovidone.

The disintegrant excipient can include an effervescent disintegrant system comprising an alkali metal or alkaline earth metal bicarbonate or carbonate, and a pharmaceutically acceptable organic acid. Preferred alkali metal and alkaline earth metal bicarbonates and carbonates are sodium carbonate, potassium carbonate, magnesium carbonate, calcium carbonate, sodium bicarbonate and potassium bicarbonate. Sodium bicarbonate is more preferred. Preferred pharmaceutically acceptable organic acids include citric acid or tartaric acid, with tartaric acid being particularly preferred. An especially preferred effervescent disintegrant system comprises sodium bicarbonate and tartaric acid.

More preferably, step (1) comprises providing a mixture of palonosetron (preferably in the form of palonosetron hydrochloride), lactose, microcrystalline cellulose, povidone and crospovidone, preferably lactose.

The mixture in step (1) can be dry mixed in a high shear mixer such as a Diosna or a low shear mixer such as a Y-cone (twin shell mixer) or similar dry blenders that provide isometric mixing. Preferably, in any preferred process of the invention, the mixing is carried out with a low shear mixer such as a Y cone mixer.

The excipients employed in step (3) preferably includes at least one of a diluent, binder and disintegrant, which may be mixed together, or preferably in stages. Preferably the mixing is in stages, with the diluent and/or mixture of diluent and binder being mixed first, followed by the disintegrant.

Further excipients may be added. Preferably, the additional excipient includes a lubricant. Thus, in any of the above processes, a lubricant may be added before the compression step (4). Preferred lubricants are selected from the group consisting of calcium stearate, glycerin monostearate, magnesium lauryl sulfate, magnesium stearate, sodium benzoate, sodium lauryl sulfate, sodium stearyl fumarate, stearic acid, talc and zinc stearate. Sodium stearyl fumarate and magnesium stearate are preferred, with sodium stearyl fumarate being particularly preferred.

The mixture is then compressed to form slugs, which are milled, preferably through a Frewitt mill to form a milled material.

After milling, further excipients may be added. The further excipients can include at least one of a diluent, disintegrant and lubricant, preferably a lubricant such as sodium stearyl fumarate. The diluent, disintegrant and lubricant are preferably selected from any of the above-mentioned diluents, disintegrants and lubricants. Preferably the diluent is selected from lactose or microcrystalline cellulose, and a combination thereof, and more preferably the diluent is lactose. Preferably the disintegrant is selected from the group consisting of crospovidone, sodium starch glycolate or croscarmellose sodium. Croscarmellose sodium is particularly preferred. Preferably the lubricant is magnesium stearate or sodium stearyl fumarate, with sodium stearyl fumarate being particularly preferred.

Preferably the resulting mixture from step (6) is filled into hard gelatin capsules, to provide a hard capsule dosage form.

In a particularly preferred process of the present invention, the process comprises:

(1) providing a mixture of palonosetron hydrochloride, diluent, and disintegrant, preferably wherein the diluent is a mixture of lactose and microcrystalline cellulose, and preferably wherein the disintegrant is a mixture of and crospovidone;

(2) dry blending the mixture in a low shear mixer;

(3) admixing the blended mixture with a lubricant, preferably sodium stearyl fumarate;

(4) compressing the mixture from step (3) into slugs;

(5) milling the slugs

(6) admixing the milled mixture with a lubricant, preferably wherein the lubricant is sodium stearyl fumarate; and

(7) filling the mixture from step (6) into hard gelatin capsules, to provide a hard capsule dosage form.

In another preferred dry granulation method in accordance with the present invention, the process comprises:

(1) providing a mixture of palonosetron hydrochloride, and diluent (preferably wherein the diluent is lactose),

(2) dry blending the mixture in a low shear mixer (e.g. a Y cone mixer);

(3) dry blending the mixture in step (2) with one or more excipients comprising at least one binder, preferably comprising a diluent and a binder (preferably wherein the binder is povidone);

(4) dry blending the mixture in step (3) in a low shear mixer with one or more excipients comprising at least one disintegrant, preferably comprising a diluent and a disintegrant (preferably wherein the disintegrant is croscarmellose sodium, or sodium starch glycolate, or a mixture thereof, and most preferably croscarmellose sodium) and;

(5) compressing the mixture from step (4) into slugs;

(6) milling the slugs

(7) admixing the milled mixture with at least one excipient, wherein the excipient preferably includes a lubricant (more preferably wherein the lubricant is sodium stearyl fumarate or magnesium stearate or a mixture thereof, and preferably sodium stearyl fumarate); and

(8) filling the mixture from step (6) into hard gelatin capsules, to provide a hard capsule dosage form.

The wet granulation method for producing the dosage form, particularly tablet formulations such as orally disintegrating tablets, of the present invention comprises the steps of:

(1) providing a mixture of at least two pharmaceutically acceptable excipients selected from the group comprising: diluent, binder, disintegrant, and combination thereof, and optionally a taste masking agent, flavouring, or sweetener or a combination thereof;

(2) blending to obtain a first mixture;

(3) granulating the first mixture with a granulation solution comprising palonosetron or a pharmaceutically acceptable salt thereof and a solvent;

(4) drying the mixture from step (3) to form a dried granulate;
(5) milling the dried granulate; and
(6) blending the milled mixture with one or more excipients, preferably wherein the excipient is selected from a glidant, a disintegrant, and a lubricant, or a combination thereof.

Optionally, the mixture from step (6) may be filled into a capsule to provide a hard capsule formulation. Preferably, the mixture is compressed into tablets.

Steps (1) and (2) may be carried out in a high or low shear mixer, preferably a high shear mixer. Steps (3)-(6) are preferably carried out in a high shear mixer.

The mixture in step (1) preferably comprises at least one diluent, at least one disintegrant and at least one binder and optionally flavouring agent and sweetener. Suitable diluents, disintegrants and binders are described above.

The one or more excipients in step (6) preferably comprises at least one diluent, disintegrant or lubricant, or a mixture thereof. Suitable diluents, disintegrants and lubricants are described above.

Preferably in this process, the mixture in step (1) comprises a diluent and a disintegrant. More preferably, the diluent is selected from the group consisting of calcium carbonate, calcium hydrogen phosphate (preferably anhydrous calcium hydrogen phosphate), cellulose, microcrystalline cellulose, ethylcellulose, magnesium carbonate, magnesium oxide, mannitol, dextrin, dextrose, sorbitol, lactose, starch, sucrose, talc, tragacanth, xylitol, and mixtures thereof. More preferably the diluent is selected from the group consisting of lactose, microcrystalline cellulose, and mixtures thereof. In a particularly preferred embodiment, the diluent comprises a combination of lactose and microcrystalline cellulose, and most preferably the diluent is lactose.

Preferably the mixture in step (1) comprises a disintegrant selected from the group consisting of alginic acid, calcium phosphate tribasic, carboxymethylcellulose sodium or calcium, cellulose, colloidal silicon dioxide, croscarmellose sodium, crospovidone, hydroxypropyl cellulose, magnesium aluminium silicate, microcrystalline cellulose, sodium starch glycolate, starch, and mixtures thereof. The preferred disintegrant in step (1) is crospovidone, sodium starch glycolate and croscarmellose sodium or a mixture thereof, and most preferably the disintegrant is sodium starch glycolate or croscarmellose sodium or a mixture thereof. Croscarmellose sodium is an especially preferred disintegrant.

In a particularly preferred process, the mixture of step (1) comprises lactose, microcrystalline cellulose, crospovidone, croscarmellose sodium and povidone, more preferably lactose, povidone and croscarmellose sodium.

In step (2), the mixture from step (1) as discussed above is granulated with a granulating solution comprising the palonosetron or pharmaceutically acceptable salt thereof. Preferably the palonosetron is in the form of palonosetron hydrochloride. The granulating solution can comprise water, a C₁₋₄ alcohol (such as ethanol) or a mixture thereof. Preferably the granulating solution is a mixture of palonosetron hydrochloride in water.

After granulating the mixture a granulate is formed. The granulate is dried, preferably in a fluid bed dryer.

The dried granulate is milled. After milling, further excipients may be added. These can include one or more of the group selected from diluent, disintegrant and lubricant. The diluent, disintegrant and lubricant are preferably selected from any of the above-mentioned diluents, disintegrants and lubricants. Preferably the diluent is selected from lactose or microcrystalline cellulose, and a combination thereof. Preferably the disintegrant is crospovidone or croscarmellose sodium, preferably croscarmellose sodium. At this stage, the disintegrant may further include an effervescent disintegrant comprising an alkali metal or alkaline earth metal bicarbonate or carbonate, and a pharmaceutically acceptable organic acid. Preferred alkali metal and alkaline earth metal bicarbonates and carbonates are sodium carbonate, potassium carbonate, magnesium carbonate, calcium carbonate, sodium bicarbonate and potassium bicarbonate. Sodium bicarbonate is more preferred. Preferred pharmaceutically acceptable organic acids include citric acid or tartaric acid, with tartaric acid being particularly preferred. An especially preferred effervescent disintegrant system comprises sodium bicarbonate and tartaric acid. Preferably the lubricant is magnesium stearate.

A particularly preferred combination of excipients in step (6) of this process is a combination of lactose, crospovidone, microcrystalline cellulose, and optionally an effervescent disintegrant system comprising sodium bicarbonate and tartaric acid, or alternatively the combination of excipients in step (6) is croscarmellose sodium and sodium stearyl fumarate.

In a preferred embodiment, the mixture from step (6) is filled into a hard gelatin capsule, or preferably, compressed into a tablet.

In a particularly preferred wet granulation process of the present invention, the process comprises:

(1) providing a mixture of lactose, microcrystalline cellulose, povidone and crospovidone

(2) dry blending to obtain a first mixture, preferably in a high shear mixer;

(3) granulating the first mixture (preferably in a high shear mixer) with a granulation solution comprising palonosetron or a pharmaceutically acceptable salt thereof and a solvent, preferably wherein the granulation solution is palonosetron hydrochloride in water;

(4) drying the mixture from step (3) to form a dried granulate;

(5) milling the dried granulate; and

(6) blending the milled mixture with a diluent, disintegrant, and a lubricant, preferably wherein the diluent is a mixture of lactose and microcrystalline cellulose, wherein the disintegrant is crospovidone optionally in combination with sodium bicarbonate and tartaric acid, and wherein the lubricant is magnesium stearate a or sodium stearyl fumarate or a mixture thereof, and preferably sodium stearyl fumarate; and

(7) filling the mixture from step (6) into a capsule to provide a hard capsule formulation.

### EXAMPLES

All % wt are relative to the total weight of the active ingredient and excipients (i.e. excluding any capsule shell or coating).

### Example 1

| | | |
|---|---|---|
| | | % in |
| | | formulation |
| Mg/tab | Ingredient | W/W |

Part I

| | | |
|---|---|---|
| 140.0 | Anhydrous Calcium Hydrogen | 56.0 |
| | Phosphate (A-Tab) | |
| 0.56* | Palonosetron HCl | 0.22 |
| 10.0 | Povidone (PVP K-30) | 4.0 |
| 10.0 | Sodium Starch Glycolate | 4.0 |
| 20.0 | Mannitol (Mannozen EZ) | 8.0 |
| 6.0 | Sodium Stearyl Fumarate | 2.4 |

Part II

| | | |
|---|---|---|
| 52.44 | Anhydrous Calcium Hydrogen | 20.98 |
| | Phosphate (A-Tab) | |
| 5.0 | Sodium Starch Glycolate | 2.0 |

Part III

| | | |
|---|---|---|
| 6.0 | Sodium Stearyl Fumarate | 2.4 |
| 250.0 | Theoretical End weight | 100.0 |

Production Method for Examples 1:
1. Components of part 1 were transferred to a high shear mixer and dry mixed.
2. The mixture from step 1 was transferred to a twin shell blender and was further mixed.
3. The mixture from step 2 was compressed into slugs.
4. The slugs from step 3 were milled through Frewitt milling machine and further transferred into a twin shell blender.
5. Components of part II were sieved and added to the mixer of step 4 and mixed.
6. Sodium Stearyl Fumarate of part III was screened and added to the mixer from step 5 and mixed to get a final blend.
7. Hard gelatin capsule shell was filled with the required quantity of final blend from step 6.

### Examples 2-3

### Wet Granulation Method

### Example 2

| | | |
|---|---|---|
| | | % in |
| | | formulation |
| Mg/tab | Ingredient | W/W |

Part I

| | | |
|---|---|---|
| 140.0 | Lactose monohydrate 200 mesh | 56.0 |
| 10.0 | Sodium starch Glycolate | 4.0 |
| 20.0 | Pregelatinized Starch | 8.0 |

Part II

| | | |
|---|---|---|
| | Granulation solution: #1 | |
| 0.56 | Palonosetron HCl | 0.22 |
| | Purified Water* | |

Part III

| | | |
|---|---|---|
| | Granulation Solution: # 2 | |
| 10.0 | Povidone (PVP K-30) | 4.0 |
| | Purified Water | |

Part IV

| | | |
|---|---|---|
| 52.44 | Lactose monohydrate 200 mesh | 20.98 |
| 5.0 | Sodium starch Glycolate | 2.0 |

Part V

| | | |
|---|---|---|
| 12.0 | Sodium Stearyl Fumarate | 4.8 |
| 250.0 | Theoretical End weight | 100.0 |

Process solvent evaporated during drying process

### Example 3

| | | |
|---|---|---|
| | | % in |
| | | formulation |
| Mg/tab | Ingredient | W/W |

Part I

| | | |
|---|---|---|
| 140.0 | Anhydrous Calcium Hydrogen | 56.0 |
| | Phosphate (A-Tab) | |
| 10.0 | Sodium starch Glycolate | 4.0 |
| 20.0 | Mannitol (Powder) | 8.0 |

Part II

| | | |
|---|---|---|
| | Granulation solution: #1 | |
| 0.56 | Palonosetron HCl | 0.22 |
| | Purified Water* | |

Part III

| | | |
|---|---|---|
| | Granulation Solution: # 2 | |
| 10.0 | Povidone (PVP K-30) | 4.0 |
| | Purified Water | |

Part IV

| | | |
|---|---|---|
| 52.44 | Anhydrous Calcium Hydrogen | 20.98 |
| | Phosphate (A-Tab) | |
| 5.0 | Sodium starch Glycolate | 2.0 |

Part V

| | | |
|---|---|---|
| 12.0 | Sodium Stearyl Fumarate | 4.8 |
| 250.0 | Theoretical End weight | 100.0 |

Production Method
1. Components of part 1 were transferred to a high shear mixer and were dry mixed.
2. Palonosetron HCl (of part II) was dissolved in purified water and added to high shear mixer from step 1 and mixed.
3. PVP K-30 (of part III) was dissolved in purified water and added to high shear mixer from step 2 and mixed to get a desired granulates.
4. The granulates from step 3 were dried in fluid bed dryer and milled through Frewitt.
5. The milled material from step 4 was transferred into a twin shell blender.
6. Components of part IV were sieved and added to the twin shell blender from step 5 and mixed.
7. Sodium stearyl Fumarate of part V was screened and add to the twin shell blender from step 6 to get a final blend.
   Hard gelatin capsule shell was filled with the required quantity of final blend from step 7.

Dissolution of the capsules made according to any of the examples in the present application was measured by using USP paddle method of 75 rpm in 500 ml 0.01N HCl at 37° C. Results are presented as weight % of the starting amount of palonosetron.

A stability test was performed on samples of Examples 1-3 after storage under a “stressed conditions” of a temperature of 55° C. and a relative humidity of 100 percent for 5 days.

A high performance liquid chromatography method (HPLC) was employed for chemical analysis using the following parameters:

### Column & Packing: ACE 5 C8, 4.6×250 mm (ACE®)

### Column Temperature: 35° C.

### Autosampler Temperature: Ambient

Mobile Phase: Acetonitrile: Buffer solution* (32:68)
Flow Rate: 1.0 mL/min
Detector: UV at 242 nm, 10-mm path length flow cell

### Injection Volume: 100 μL

### Diluent 1: Water:Acetonitrile (50:50)

### Diluent 2: 0.01N HCl:Acetonitrile (80:20)

Blank: Diluent 2 passed through an intended for work filter

### Injector Wash Solution: Diluent 1

Dissolution and Stability results are shown in the Table below:

| | % dissolution (% RDS) | | |
|---|---|---|---|
| Time (min) | Example 1 | Example 2 | Example 3 |
| 15 | 33 (33.0) | 80 (5.6) | 42 (35.0) |
| 30 | 63 (27.8) | 94 (3.0) | 60 (27.4) |
| 45 | 76 (26.7) | 98 (2.8) | 68 (21.5) |
| 60 | 83 (23.1) | 102 (2.7) | 73 (18.2) |
| Total degradant* | <0.15% | <1% | <0.7% |

*Total degradant was calculated as percent area.

### Example 4

| | | |
|---|---|---|
| | | % in |
| | | formulation |
| Mg/tab | Ingredient | W/W |

Part I

| | | |
|---|---|---|
| 80.0 | Lactose monohydrate (Lactose DCL 14) | 40.0 |
| 0.56* | Palonosetron HCl | 0.28 |
| 56.44 | Lactose monohydrate 100 mesh | 28.22 |
| 20.0 | Sodium Starch Glycolate | 10.0 |

Part II

| | | |
|---|---|---|
| 2.0 | Sodium Stearyl Fumarate | 1.0 |

Part III

| | | |
|---|---|---|
| 20.0 | Pregelatinized Starch | 10.0 |
| 20.0 | Croscarmellose Sodium | 10.0 |

Part IV

| | | |
|---|---|---|
| 1.0 | Magnesium Stearate | 0.5 |
| 200.0 | Theoretical End weight | 100.0 |

Preparation Method of Example 4
1. Components of part I were transferred in to high shear mixer and dry mixed.
2. Mix from step 1 was transferred in to a twin shell blender from step 1 and mixed
3. Component of part II was screened (through sieve #50 mesh) and then added to step 2 and mixed
4. Mix from step 3 was compressed in to slugs.
5. Slugs from step 4 were milled through Frewitt milling machine.
6. Milled material from step 5 was transferred in to twin shell blender.
7. Components of part III were sieved through #30 mesh, added to the twin shell blender from step 6 and mixed.
8. Magnesium Stearate of part IV was screened through #50 mesh, added to twin shell blender from step 7 and mixed to get the final blend
9. Required quantity of final blend from step 8 was filled in to hard gelatin capsules.

| Time (min) | % dissolution (% RDS) |
|---|---|
| 15 | 89 (0.6) |
| 30 | 89 (0.6) |
| 45 | 89 (0.5) |
| 60 | 89 (0.4) |
| % Blend Uniformity | 89.8 (6.6) |

### Example 5

### Orally Disintegrating Tablets

| | | |
|---|---|---|
| | | % in |
| | | formulation |
| Mg/Capsule | Ingredients | W/W |

Part I

| | | |
|---|---|---|
| 80.0 | Mannitol | 40.0 |
| 0.5 | Artificial Cherry Flavor | 0.25 |
| 10.0 | Croscarmellose Sodium N594 S.D | 5.0 |
| 5.0 | Saccharin Sodium | 2.5 |
| 82.44 | Lactose Monohydrate | 41.22 |
| 8.0 | Pregelatinized Starch | 4.0 |
| | Granulation medium: | |
| 0.56* | Palonosetron HCl | 0.28 |
| | Purified Water** | — |
| | Purified Water** (q.s.) | — |

Part II

| | | |
|---|---|---|
| 0.5 | Colloidal Silicon Dioxide | 0.25 |

Part III

| | | |
|---|---|---|
| 10.0 | Croscarmellose Sodium | 5.0 |

Part IV

| | | |
|---|---|---|
| 3.0 | Sodium Stearyl Fumarate | 1.5 |
| 200.0 | Theoretical End Weight | 100.0 |

*0.56 mg Palonosetron HCl is equivalent to 0.5 mg Palonosetron Base
**Granulation medium

Production Method (Wet Granulation)
1. Transfer the components of part Tin to high shear mixer and mix
2. Prepare granulation solution by dissolving Palonosetron HCl in purified water
3. Add granulation solution from step 2 to high shear mixer form step 1 and mix to get the desired granulate
4. Dry the granulate from step 3 in fluid bed dryer
5. Add the component of part II to dried granulate from step 4.
6. Mill the dried granulate from step 5 through Frewitt milling machine/comill or Fitz mill.
7. Transfer the milled granulate from step 6 in to twin-shell blender or flow bin and mix
8. Sieve (through #30 mesh) and add the components of part III to mixer from step 7 and mix
9. Sieve the component of part IV through #50 mesh, add to mixer from step 8 and mix to get the final blend
10. Compress the final blend from step 9 in to tablets

### Example 6

### Dry Mix Method

| | | |
|---|---|---|
| | | % in |
| | | formulation |
| Mg/Capsule | Ingredients | W/W |

Part I

| | | |
|---|---|---|
| 15.0 | Lactose Monohydrate (100 mesh) | 7.5 |
| 15.0 | Lactose Monohydrate (DCL 14) | 7.5 |

Part II

| | | |
|---|---|---|
| 0.56* | Palonosetron HCl | 0.28 |
| 6.44 | Lactose Monohydrate 200 mesh | 3.22 |

Part III

| | | |
|---|---|---|
| 15.0 | Lactose Monohydrate (100 mesh) | 7.5 |
| 15.0 | Lactose Monohydrate (DCL 14) | 7.5 |

Part IV

| | | |
|---|---|---|
| 30.0 | Lactose Monohydrate (100 mesh) | 15.0 |
| 30.0 | Lactose Monohydrate (DCL 14) | 15.0 |
| 10.0 | Povidone (PVP K-30) | 5.0 |

Part V

| | | |
|---|---|---|
| 20.0 | Lactose Monohydrate (100 mesh | 10.0 |
| 20.0 | Lactose Monohydrate (DCL 14) | 10.0 |
| 20.0 | Croscarmellose Sodium | 10.0 |

Part VI

| | | |
|---|---|---|
| 3.0 | Sodium Stearyl Fumarate | 1.5 |
| 200.0 | Theoretical End Weight | 100.0 |

### Production Method (Direct Compression):

Final blend of direct compression method is arrived at by using geometrical mixing method. Percentage of API in the formulation being very small, it is first mixed with a small amount of excipient to perform trituration (part II) and is then premixed with about one third of the remaining quantity of excipients (part II and part III). This premix is then added to the remainder of the excipients (part IV and part V) and is sieved through Co-mill. This mix is then transferred to the twin shell or tumbler blender and is mixed. And finally lubricant is added to the mix and is blended to get the final blend. This final blend is then filled in to capsule.
1. Sieve (through #30 mesh) and transfer the components of part I to twin shell mixer or tumbler mixer
2. Add lactose of part II to polyethylene bag containing Palonosetron HCl and perform trituration.
3. Sieve the mix from step 2 through #50 mesh and transfer to mixer from step 1
4. Sieve (through #30 mesh) and transfer the components of part III to twin shell mixer or tumbler mixer and mix
5. Empty the mix from step 4 in to container
6. Sieve (through #30 mesh) and transfer the components of part IV to twin shell mixer or tumbler mixer
7. Add mix from step 5 to mixer from step 6
8. Sieve (through #30 mesh) and transfer the components of part V to mixer from step 6 and mix
9. Sieve (through #50 mesh) the component of part VI, add to mixer from step 8 and mix to get the final blend.
10. Fill the required quantity of final blend from step 13 in to suitable size of hard gelatin capsule shell, or compress into a tablet.

### Example 7-8

### Dry Granulation Method

### Example 7

| | | |
|---|---|---|
| | | % in |
| | | formulation |
| Mg/Capsule | Ingredients | W/W |

Part I

| | | |
|---|---|---|
| 10.0 | Lactose Monohydrate (100 mesh) | 5.0 |
| 10.0 | Lactose Monohydrate (DCL 14) | 5.0 |

Part II

| | | |
|---|---|---|
| 0.56* | Palonosetron HCl | 0.28 |
| 5.0 | Lactose Monohydrate 200 mesh | 2.5 |

Part III

| | | |
|---|---|---|
| 10.0 | Lactose Monohydrate (100 mesh) | 5.0 |
| 10.0 | Lactose Monohydrate (DCL 14) | 5.0 |

Part IV

| | | |
|---|---|---|
| 20.0 | Lactose Monohydrate (100 mesh) | 10.0 |
| 30.0 | Lactose Monohydrate (DCL 14) | 15.0 |
| 10.0 | Pregelatinized Starch | 5.0 |

Part V

| | | |
|---|---|---|
| 21.44 | Lactose Monohydrate (100 mesh | 10.72 |
| 30.0 | Lactose Monohydrate (DCL 14) | 15.0 |
| 20.0 | Sodium Starch Glycolate | 10.0 |

Part VI

| | | |
|---|---|---|
| 2.0 | Sodium Stearyl Fumarate | 1.0 |

Part VII

| | | |
|---|---|---|
| 20.0 | Croscarmellose Sodium | 10.0 |

Part VIII

| | | |
|---|---|---|
| 1.0 | Magnesium Stearate | 0.5 |
| 200.0 | Theoretical End Weight | 100.0 |

Production Method for Example 7:
1. Ingredients of Part I were sieved (through #50 mesh) and transferred to a twin shell mixer.
2. Lactose of part II was added to polyethylene bag containing Palonosetron HCl and trituration was performed.
3. The mixture from step 2 was sieved (through #50 mesh) and added to the mixer from step 1.
4. Ingredients of Part III were sieved (through #50 mesh) and transferred to the mixer from step 1 and mixed.
5. The mixture from part 4 was emptied into a container.
6. Ingredients of Part IV were sieved (through #30 mesh) and transferred to a twin shell mixer.
7. The mixture from step 5 was added to the mixer from step 6.
8. Ingredients of Part V were sieved (through #30 mesh) and transferred to the mixer from step 6 and mixed.
9. Ingredients of Part VI were sieved (through #50 mesh) and transferred to the mixer from step 8 and mixed to get the blend ready for compression into “slugs”.
10. The blend from step 9 was compressed into “slugs”.
11. The slugs from step 10 were milled through Frewit and transferred to a twin shell mixer.
12. Ingredients of Part VII were sieved (through #30 mesh) and transferred to the mixer from step 11 and mixed.
13. Ingredients of Part VIII were sieved (through #50 mesh) and transferred to the mixer from step 12 and mixed to get the final blend.
14. A required quantity of final blend from step 13 was filled into a hard gelatin capsule shell.

### Example 8

| | | |
|---|---|---|
| | | % in |
| | | formulation |
| Mg/Capsule | Ingredients | W/W |

Part I

| | | |
|---|---|---|
| 15.0 | Lactose Monohydrate (100 mesh) | 7.5 |
| 15.0 | Lactose Monohydrate (DCL 14) | 7.5 |

Part II

| | | |
|---|---|---|
| 0.56* | Palonosetron HCl | 0.28 |
| 6.44 | Lactose Monohydrate 200 mesh | 3.22 |

Part III

| | | |
|---|---|---|
| 15.0 | Lactose Monohydrate (100 mesh) | 7.5 |
| 15.0 | Lactose Monohydrate (DCL 14) | 7.5 |

Part IV

| | | |
|---|---|---|
| 30.0 | Lactose Monohydrate (100 mesh) | 15.0 |
| 30.0 | Lactose Monohydrate (DCL 14) | 15.0 |
| 10.0 | Povidone (PVP K-30) | 5.0 |

Part V

| | | |
|---|---|---|
| 20.0 | Lactose Monohydrate (100 mesh | 10.0 |
| 20.0 | Lactose Monohydrate (DCL 14) | 10.0 |
| 20.0 | Croscarmellose Sodium | 10.0 |

Part VI

| | | |
|---|---|---|
| 2.0 | Sodium Stearyl Fumarate | 1.0 |

Part VII

| | | |
|---|---|---|
| 1.0 | Sodium Stearyl Fumarate | 0.5 |
| 200.0 | Theoretical End Weight | 100.0 |

Production Method for Example 8:
1. Ingredients of Part I were sieved (through #50 mesh) and transferred to a twin shell mixer.
2. Lactose of part II was added to polyethylene bag containing Palonosetron HCl and trituration was performed.
3. The mixture from step 2 was sieved (through #50 mesh) and added to the mixer from step 1.
4. Ingredients of Part III were sieved (through #50 mesh) and transferred to the mixer from step 1 and mixed.
5. The mixture from part 4 was emptied into a container.
6. Ingredients of Part IV were sieved (through #30 mesh) and transferred to a twin shell mixer.
7. The mixture from step 5 was added to the mixer from step 6.
8. Ingredients of Part V were sieved (through #30 mesh) and transferred to the mixer from step 6 and mixed.
9. Ingredients of Part VI were sieved (through #50 mesh) and transferred to the mixer from step 8 and mixed to get the blend ready for compression into “slugs”.
10. The blend from step 9 was compressed into “slugs”.
11. The slugs from step 10 were milled through Frewit and transferred to a twin shell mixer and mixed
12. Ingredients of Part VII were sieved (through #50 mesh) and transferred to the mixer from step 11 and mixed to get the final blend.
13. A required quantity of final blend from step 12 was filled into a hard gelatin capsule shell.

### Dissolution Test

Dissolution of the preparations made according to Examples 7 and 8 was measured by using USP paddle method of 75 rpm in 500 ml 0.01N HCl at pH 2 at 37° C.

| Time (min) | % dissolution (% RDS) |
|---|---|
| 15 | 98 (1.0) |
| 30 | 99 (1.0) |
| 45 | 99 (0.8) |
| 60 | 99 (0.8) |
| % Blend Uniformity | 95.3 (0.6) |

Dissolution of 12 capsules prepared according to example 8 was measured by using USP paddle method of 75 rpm in 500 ml 0.01N HCl at pH 2 at 37° C.

| Time (minutes) | Average dissolution | R.S.D (%) |
|---|---|---|
| 15 | 97 | 3.4 |
| 30 | 98 | 3 |
| 45 | 98 | 2.8 |

### Analysis of Blend Uniformity and Content Uniformity—Example 8

### Analytical Method for Uniformity of Content by HPLC

### A. Blend Uniformity Sampling

1. For blend uniformity determination, ten samples, each one equivalent to the approximate weight of one capsule fill weight (200 mg) was collected from the mixer at a range of locations (e.g. for Y-cone, samples are taken from left and right arms and bottom). Twenty additional supporting samples are collected in the same procedure and reserved for further testing if required. The ten samples are assayed in their entirety for blend uniformity by the procedure discussed below.

2. For blend uniformity determination, ten samples, each one equivalent to the approximate weight of one capsule fill weight (200 mg) was collected from the storage container at a range of locations (e.g. 3 samples from top, 3 samples from bottom and 4 samples from middle). Twenty additional supporting samples are collected in the same procedure and reserved for further testing if required. The samples are assayed for content uniformity by the following procedure.

### B. Content Uniformity Sampling

For content uniformity determination, ten capsules were assayed for content uniformity by the following procedure.

### C. Determination of Blend/Content Uniformity

Content of palonosetron was calculated (% of label claim) in each capsule taken by formula:

$$\frac{Smp . peak area \times Stock std . conc {.}^{*} ( mg \text{/} mL ) \times {0.89}^{**}}{Work . std . avg . peak area} \times 200 = \\ {}^{*} Take into account the \% assay and the \% water of the relevant$$

$$standard . \\ {}^{**} The ratio of the molecular weight of palonosetron to that of$$

$$palonosetron HCl ( 296.41 / 332.87 ) .$$

And then acceptance value or range was calculated.

### Analytical Method for Uniformity of Blend by HPLC

The % of declared amount of palonosetron was calculated by the formula:

$$\frac{\begin{matrix}Smp . peak area \times Stock std . conc {.}^{*} ( mg \text{/} mL ) \times \\ Theor . wt . of caps . contents ( mg ) \times Smp . vol . ( mL ) \times {0.89}^{**}\end{matrix}}{Work . std . avg . peak area \times Smp . wt . ( mg )} \times 4$$

$${}^{*} Take into the account the \% assay and the \% water of the standard . \\ {}^{**} The ratio of the molecular weight of palonosetron to that of$$

$$palonosetron HCl ( 296.41 / 332.87 )$$

Content uniformity was analyzed by HPLC method as described in example 3.

Blend uniformity was analyzed by HPLC method, which employs a reverse phase ACE 5 C8, 4.6×50 mm (ACE®) column, operating at temperature 40° C., UV detection at 242 nm and a mixture of 30 mM anhydrous dibasic sodium phosphate (Na₂HPO₄) and 30 mM of sodium perchlorate (NaClO₄.H2O) buffer solution at pH of a 3.0 and Acetonitrile in ratio 68:32 as mobile phase.

Sample analysis was conducted on both the blend and final dosage form.

| | |
|---|---|
| Results of Blend Uniformity measurements | Average (%): 97.4 |
| from storage container (n = 10): | R.S.D (%): 2.4 |
| Results of Blend Uniformity measurements | Average (%): 97.2 |
| from Y-Cone (n = 10): | R.S.D (%): 1.9 |
| Results of Content Uniformity measurements | Average (%): 98.2 |
| (n = 10): | RSD (%): 2.8 |

A stability test was performed on samples of Example 8 initially after they were prepared and after storage under the accelerated conditions of a temperature of 40° C. and a relative humidity of 75 percent for 1, 2 and 3 months.

A high performance liquid chromatography method was employed for chemical analysis using the following parameters:

### Column & Packing: ACE 5 C8, 4.6×250 mm (ACE®)

### Column Temperature: 35° C.

### Autosampler Temperature: Ambient

Mobile Phase: Acetonitrile:Buffer solution* (32:68)
Flow Rate: 1.0 mL/min
Detector: UV at 242 nm, 10-mm path length flow cell

### Injection Volume: 100 μL

### Diluent 1: Water:Acetonitrile (50:50)

### Diluent 2: 0.01N HCl:Acetonitrile (80:20)

Blank: Diluent 2 passed through an intended for work filter

### Injector Wash Solution: Diluent 1

### Stability Results

| | 0M | | | | |
|---|---|---|---|---|---|
| Pack type | ASSAY | DISS | N-OXID | UNKNU | TOTAL |
| Bot. 100 CC*100 Caps. CRC | 98.6 | 98 | <0.1 | <0.1 | <0.1 |
| Bot. 100 CC*100 Caps. PP | 98.6 | 98 | <0.1 | <0.1 | <0.1 |
| Bot. 40 CC*5 Caps. CRC | 98.6 | 98 | <0.1 | <0.1 | <0.1 |
| Bot. 750 CC*1000 Caps. PP | 98.6 | 98 | <0.1 | <0.1 | <0.1 |
| Bot. 60 CC*5 Caps. CRC | 98.6 | 98 | <0.1 | <0.1 | <0.1 |
| Blister PVDC/ALUM | 98.6 | 98 | <0.1 | <0.1 | <0.1 |

| | 1M | | | | | |
|---|---|---|---|---|---|---|
| | | | | | | Relative |
| Pack type | ASSAY | DISS | N-OXID | UNKNU | TOTAL | Assay |
| Bot. 100 CC*100 Caps. CRC | 99.2 | 103 | <0.1 | <0.1 | <0.1 | 100.61 |
| Bot. 100 CC*100 Caps. PP | 98.3 | 105 | <0.1 | <0.1 | <0.1 | 99.70 |
| Bot. 40 CC*5 Caps. CRC | 99.5 | 103 | <0.1 | <0.1 | <0.1 | 100.91 |
| Bot. 750 CC*1000 Caps. PP | 99 | 102 | <0.1 | <0.1 | <0.1 | 100.41 |
| Bot. 60 CC*5 Caps. CRC | 98.1 | 102 | <0.1 | <0.1 | <0.1 | 99.49 |
| Blister PVDC/ALUM | 99.4 | 99 | <0.1 | <0.1 | <0.1 | 100.81 |
| | 2M | | | | | |
| | | | | | | Relative |
| Pack type | ASSAY | DISS | N-OXID | UNKNU | TOTAL | Assay |
| Bot. 100 CC*100 Caps. CRC | 99.4 | 100 | <0.1 | <0.1 | <0.1 | 100.81 |
| Bot. 100 CC*100 Caps. PP | 98.3 | 100 | <0.1 | <0.1 | <0.1 | 99.70 |
| Bot. 40 CC*5 Caps. CRC | 98.6 | 96 | <0.1 | <0.1 | <0.1 | 100.00 |
| Bot. 750 CC*1000 Caps. PP | 97.9 | 97 | <0.1 | <0.1 | <0.1 | 99.29 |
| Bot. 60 CC*5 Caps. CRC | 98.1 | 99 | <0.1 | <0.1 | <0.1 | 99.49 |
| Blister PVDC/ALUM | 97.6 | 97 | <0.1 | <0.1 | <0.1 | 98.99 |
| | 3M | | | | | |
| | | | | | | Relative |
| Pack type | ASSAY | DISS | N-OXID | UNKNU | TOTAL | Assay |
| Bot. 100 CC*100 Caps. CRC | 96.5 | 100 | <0.1 | <0.1 | <0.1 | 97.87 |
| Bot. 100 CC*100 Caps. PP | 98 | 100 | <0.1 | <0.1 | <0.1 | 99.39 |
| Bot. 40 CC*5 Caps. CRC | 96.3 | 99 | <0.1 | <0.1 | <0.1 | 97.67 |
| Bot. 750 CC*1000 Caps. PP | 97 | 97 | <0.1 | <0.1 | <0.1 | 98.38 |
| Bot. 60 CC*5 Caps. CRC | 97.7 | 101 | <0.1 | <0.1 | <0.1 | 99.09 |
| Blister PVDC/ALUM | 98.2 | 99 | <0.1 | <0.1 | <0.1 | 99.59 |

It can be concluded from the stability results of Palonosetron HCl capsules under conditions of 40° C. and 75% RH that Palonosetron HCl capsule of the present invention are stable. No increase in N-oxide or any unknown impurity was observed at the end of 3 months under stability conditions of 40° C. and 75% RH.

1. A dosage form for oral administration comprising a solid admixture of palonosetron or a pharmaceutically acceptable salt thereof and at least one pharmaceutically acceptable excipient.

2. A dosage form according to claim 1 wherein the solid admixture has a blend uniformity of about 90% to about 110% with an associated relative standard deviation of about 5% or less; and/or a content uniformity of about 90% to about 110% with an associated relative standard deviation of about 5% or less, as measured by the concentration of palonosetron or pharmaceutically acceptable salt thereof.

3. A dosage form according to claim 1 wherein the solid admixture is prepared by dry granulation.

4. A dosage form according to claim 1 wherein the solid admixture is prepared in a low shear mixer.

5. A dosage form according to claim 1 wherein about 1 wt % or less, based on the initial weight of palonosetron at the start of the storage, palonosetron N-oxide is present in the dosage form after storage of the dosage form for 90 days at 40° C. and 75% relative humidity.

6. A dosage form according to claim 1 wherein about 0.5 wt % or less, based on the initial weight of palonosetron at the start of the storage, palonosetron N-oxide is present in the dosage form after storage of the dosage form for 90 days at 40° C. and 75% relative humidity.

7. A dosage form according to claim 1 wherein the pharmaceutically acceptable salt of palonosetron is palonosetron hydrochloride.

8. A dosage form according to claim 1 wherein the palonosetron or pharmaceutically acceptable salt thereof is present in an amount of between about 0.02 mg to about 10 mg per dosage form based on the weight of palonosetron base.

9. A dosage form according to claims 1 wherein the palonosetron or pharmaceutically acceptable salt thereof is present in an amount of between about 0.1 mg to about 5 mg per dosage form based on the weight of palonosetron base.

10. A dosage form according to claim 1 wherein the palonosetron is present in an amount of about 0.25 mg, about 0.5 mg or about 0.75 mg per dosage form based on the weight of palonosetron base.

11. A dosage form according to claim 1 wherein the palonosetron or pharmaceutically acceptable salt thereof is present in a concentration of about 0.05 wt % to about 5 wt %, wherein the wt % is relative to the weight of the dosage form excluding any tablet coating and capsule shell.

12. A dosage form according to claim 1 in the form of a powder.

13. A dosage form according to claim 1 wherein the solid admixture is in the form of a compressed tablet or an effervescent tablet.

14. A dosage form according to claim 1 in the form of a filled capsule or a sprinkle formulation.

15. A dosage form according to claim 14 in the form of a filled hard gelatin capsule.

16. A dosage form according to claim 1 in the form of a capsule comprising:
(a) a gelatin outer shell, and
(b) a solid admixture of palonosetron or a pharmaceutically acceptable salt thereof with at least one pharmaceutically acceptable excipient.

17. A dosage form according to claim 1 wherein the at least one pharmaceutically acceptable excipient is selected from the group consisting of binders, disintegrants, diluents, and lubricants.

18. A dosage form according to claim 1 wherein the pharmaceutically acceptable excipient is:
(1) a combination of at least one binder, at least one disintegrant, at least one diluent, and at least one lubricant;
(2) a combination of at least one disintegrant, at least one diluent, at least one lubricant, and at least one glidant; or
(3) a combination of at least one disintegrant, at least one diluent, and at least one lubricant.

19. A dosage form according to claim 17 wherein the binder is selected from the group consisting of gelatin, cellulose, cellulose derivatives, polyvinylpyrrolidone (povidone), starch, sucrose, polyethylene glycol xylitol, sorbitol, and maltitol.

20. A dosage form according to claim 17 wherein the binder is povidone.

21. A dosage form according to claim 17 wherein the binder is povidone having a K-value of about 27 to about 32.

22. A dosage form according to claim 17 wherein the binder is povidone K30.

23. A dosage form according to claim 17 wherein the binder is present in an amount of about 1 wt % to about 10 wt % based on the weight of the dosage form excluding any capsule shell or tablet coating.

24. A dosage form according to claim 17, wherein the disintegrant is selected from the group consisting of alginic acid, carboxymethylcellulose sodium or calcium, cellulose, colloidal silicon dioxide, croscarmellose sodium, crospovidone, hydroxypropyl cellulose, magnesium aluminium silicate, microcrystalline cellulose, sodium bicarbonate, sodium starch glycolate, pregelatinized starch, tartaric acid, citric acid, and mixtures thereof.

25. A dosage form according to claim 17 wherein the disintegrant is selected from the group consisting of pregelatinized starch, crospovidone, and a mixture of sodium bicarbonate and tartaric acid.

26. A dosage form according to claim 17 wherein the disintegrant is selected from pregelatinized starch, sodium starch glycolate, croscarmellose sodium, and mixtures thereof.

27. A dosage form according to claim 17 wherein the disintegrant is present in an amount of about 5 to about 35 wt % of the dosage form, excluding any capsule shell or tablet coating.

28. A dosage form according to claim 17 wherein the disintegrant is present in an amount of about 8 to about 25 wt % of the dosage form excluding any capsule shell or tablet coating.

29. A dosage form according to claim 17 wherein the disintegrant is present in an amount of about 5 to about 15 wt % of the dosage form, excluding any capsule shell or tablet coating.

30. A dosage form according to claim 29 in the form of a filled hard gelatine capsule wherein the disintegrant is croscarmellose sodium, sodium starch glycolate, or a mixture thereof.

31. A dosage form according to claim 30 wherein the disintegrant is croscarmellose sodium present in an amount of about 8-12 wt % of the dosage form excluding the capsule shell.

32. A dosage form according to claim 17 wherein the diluent is selected from the group consisting of lactose, calcium carbonate, calcium hydrogen phosphate, cellulose, microcrystalline cellulose, ethylcellulose, magnesium carbonate, magnesium oxide, mannitol, dextrin, dextrose, sorbitol, starch, sucrose, talc, tragacanth, xylitol, and mixtures thereof.

33. A dosage form according to claim 17 wherein the diluent is selected from lactose, microcrystalline cellulose, mannitol, starch, calcium hydrogen phosphate, and mixtures thereof.

34. A dosage form according to claim 30 wherein calcium hydrogen phosphate is included in an amount of less than about 70 wt %, excluding any capsule shell or tablet coating.

35. A dosage form according to claim 17 wherein the diluent is selected from lactose, microcrystalline cellulose, mannitol, starch, and mixtures thereof.

36. A dosage form according to claim 17 wherein the diluent is selected from the group consisting of lactose, microcrystalline cellulose, mannitol, and mixtures thereof.

37. A dosage form according to claim 35 wherein the diluent is lactose.

38. A dosage form according to claim 37 wherein the lactose comprises crystalline alpha lactose monohydrate, amorphous lactose, or a mixtures thereof.

39. A dosage form according to claim 38 wherein the lactose is lactose 100 mesh, lactose 200 mesh, spray dried lactose, and mixtures thereof.

40. A dosage form according to claim 17 wherein the diluent is present in an amount of about 30 wt % to about 90 wt %, excluding any capsule shell or tablet coating.

41. A dosage form according to claim 17 wherein the lubricant is selected from the group consisting of calcium stearate, glycerin monostearate, magnesium lauryl sulfate, magnesium stearate, sodium benzoate, sodium lauryl sulfate, sodium stearyl fumarate, stearic acid, talc, zinc stearate, and mixtures thereof.

42. A dosage form according to claim 17 wherein the lubricant is selected from the group consisting of magnesium stearate, sodium stearyl fumarate, and mixtures thereof.

43. A dosage form according to claim 17 wherein the lubricant is present in an amount of about 0.1 wt % to about 6.0 wt %, excluding any capsule shell or tablet coating.

44. A dosage form according to claim 17 wherein the lubricant is present in an amount of about 0.5 wt % to about 2.5 wt %, excluding any capsule shell or tablet coating.

45. A dosage form according to claim 18 wherein the glidant is colloidal silicon dioxide.

46. A dosage form according to claim 1 in the form of a compressed tablet.

47. A dosage form according to claim 1 in the form an orally disintegrating tablet.

48. A dosage form according to claim 47 wherein a disintegrant is present in an amount of about 5 wt % to about 25 wt % based on the weight of the dosage form excluding any tablet coating.

49. A dosage form according to claim 48 wherein the disintegrant is croscarmellose sodium, pregelatinized starch, or a mixture thereof.

50. A dosage form according to claim 47 comprising a diluent selected from lactose, mannitol, and a mixture thereof.

51. A dosage form according to claim 47 comprising colloidal silicon dioxide in an amount of about 0.1 to about 0.5 wt % based on the weight of the dosage form, excluding any tablet coating.

52. A dosage form according to claim 17 in the form of a filled hard gelatin capsule wherein the disintegrant is crospovidone.

53. A dosage form according to claim 17 in the form of an orally disintegrating tablet or effervescent tablet wherein the disintegrant is selected from crospovidone, an effervescent disintegrant, and a mixtures thereof.

54. A dosage form according to claim 53, wherein the dosage form comprises an effervescent disintegrant, and wherein the effervescent disintegrant is a mixture of sodium bicarbonate and tartaric acid.

55. A dosage form according to claim 54, wherein the weight ratio between sodium bicarbonate and tartaric acid is about 2:1 to about 1:2.

56. A dosage form according to claim 54 wherein the sodium bicarbonate and tartaric acid is present in a combined amount of about 2 to about 20 wt % based on the weight of the dosage form, excluding any tablet coating.

57. A hard gelatin capsule or compressed tablet dosage form comprising, based on the total weight of the dosage form, excluding any capsule shell and tablet coating:
(1) palonosetron hydrochloride in an amount of about 0.1 to about 2.0 wt %;
(2) diluent in an amount of about 60 to about 85 wt %;
(3) binder in an amount of about 1 to about 8 wt %;
(4) disintegrant in an amount of about 5 to about 25 wt %; and
(5) lubricant in an amount of about 0.5 to about 2 wt %.

58-62. (canceled)

63. An orally disintegrating tablet or effervescent dosage form, based on the total weight of the tablet, excluding any coating:
palonosetron hydrochloride in an amount of about 0.1 to about 2.0 wt %;
diluent in an amount of about 60 to about 80 wt %;
binder in an amount of about 1 to about 8 wt %;
disintegrant in an amount of about 15 to about 30 wt %; and
lubricant in an amount of about 0.5 to about 1.5 wt %.

64. A dosage form according to claim 1 which does not contain any antioxidant.

65. A process for the preparation of a dosage form of claim 1, comprising direct compression, dry granulation, or wet granulation of the palonosetron or a pharmaceutically acceptable salt thereof and the at least one pharmaceutically acceptable excipient.

66-95. (canceled)

96. A dosage form according to claim 1 wherein, after storage of the dosage form for 90 days at 40° C. and 75% relative humidity, any increase in the amount of palonosetron N-oxide present in the dosage form is about 1 wt % or less, wherein the wt % is based on the initial weight of palonosetron or a pharmaceutically acceptable salt thereof at the start of the storage.

97. The dosage form of claim 96, wherein the increase in the amount of palonosetron N-oxide present in the dosage form is about 0 wt % after the storage.

98. (canceled)

99. (canceled)

100. The dosage form of claim 13, wherein the solid admixture is in the form of a compressed tablet.

101. The dosage form of claim 100, wherein the tablet is coated.

102. A dosage form according to claim 39, wherein the lactose is a mixture of alpha lactose monohydrate and amorphous lactose.

103. A dosage form according to claim 52, wherein the crospovidone is present in an amount of about 10 to about 20 wt % based on the weight of the dosage form, excluding any capsule shell.
